(19) United States
(12) Patent Application Publication
(10) Pub. No.: US 2010/0136080 A1
(43) Pub. Date: Jun. 3, 2010

(54) MATERIAL DEPOT FOR RELEASING AN ANTIBACTERIAL ACTIVE AGENT

(76) Inventor: Dietmar Wolter, Hoisdorf (DE)

Correspondence Address: MORRISON & FOERSTER LLP, 1650 TYSONS BOULEVARD, SUITE 400, MCLEAN, VA, 22102, US

(21) Appl. No.: 12/520,810

(22) PCT Filed: Dec. 11, 2007
(86) PCT No.: PCT/EP2007/010820

§ 371 Date: Jan. 21, 2010

(30) Foreign Application Priority Data
Dec. 20, 2006 (DE) 10 2006 060 938.7

(51) Int. Cl. A61K 9/00; A61K 31/7036; A61P 31/00

(52) U.S. Cl. 424/422; 514/40

(57) ABSTRACT

The subject matter of the invention is a material depot which dispenses an antibacterial active ingredient material into a human or animal body via the surface of the depot. The surface of the material depot is larger than the envelope of the material depot. The increased surface of the material depot effects a higher level of the antibacterial active ingredient material at the location of the implant and permits effective destruction in situ of both sensitive pathological germs and partially resistant germs.

### REFERENCE TO RELATED APPLICATIONS

This application is a national stage application under 35 USC 371 of International Application No. PCT/EP2007/010820, filed Dec. 11, 2007, which claims the priority of German Patent Application No. 10 2006 060 938.7, filed Dec. 20, 2006, the contents of which prior applications are incorporated herein by reference.

### FIELD OF THE INVENTION

The invention relates to a material depot for dispensing an antibacterial active ingredient material in a human or animal body.

### BACKGROUND OF THE INVENTION

Every surgical intervention leads to contamination of the wound with germs. If the treating medical practitioner or the carer does not, for example, wear sterile gloves and does not effect a sufficient disinfection, this can result in contamination with pathological germs, which populate the wound.

Material depots, which dispense the antibiotic to the surroundings, e.g. the wound secretion, and are thus intended to prevent or fight an infection, are known from disclosed previous use. Gentamycin-poly (methyl methacrylate) (PMMA) chains are mentioned in an exemplary manner; these are also known as Septopal® chains to a person skilled in the art. These Septopal® chains have become the therapeutic standard in osteomyelitis therapy and are also used for infection prevention. The acryl globules, infused with the antibiotic gentamycin and fixed on a wire, are intended to fight the infection or prevent it in situ by continually dispensing the antibiotic.

Compared to a systemic antibiotics therapy, Septopal® chains can obtain higher active ingredient concentrations at the location of the infection. It is known that the destruction of the germs is not only dependent on the presence of the antibiotic in the environment surrounding the bacterium, but also on the local concentration of the antibiotic (antibiotics level). The treatment with Septopal® chains is often still effective if microbiological tests verify a “simple” resistance to antibiotics, since bacteriological testing usually only examines such concentrations of antibiotics which can be attained during the course of an intravenous therapy.

It is known that even if antibiotics-containing material depots of the type mentioned initially are used, it nevertheless often is the case that not enough antibiotics are dispensed in order to ensure a high level of antibiotics. In unfavorable circumstances, when the antibiotic has been completely washed out, the antibiotics carrier can on the contrary even become a bed for bacteria to populate.

This is attempted to be precluded by, depending on the strength of the infection events, implanting a different number of spheres or Septopal® chains, or larger spheres, in order to maintain a sufficiently high level of antibiotics. However, the insertion of a plurality of spheres or Septopal® chains, or large spheres, in the wound is bound by spatial limits. In any case, an expansion of the surgical measure is necessary. This is a significant disadvantage because the patient is additionally adversely affected by this measure. Likewise, the handling when using a multiplicity of spheres or Septopal® chains is complicated, because each sphere or chain is intended to be inserted and explanted at the end of the therapy.

### SUMMARY OF THE INVENTION

It is the object of the invention to create a material depot of the type mentioned initially which does not have the disadvantages mentioned above.

In accordance with claim 1, the surface of the material depot, via which the antibacterial active ingredient material is dispensed into the human or animal body, is larger than the envelope of the material depot.

The envelope describes a closed surface spanned around a body. In a cross-sectional view, it holds true that the envelope touches each curve of a family of curves at one point. By way of example, the envelope of a sphere with a base radius r and grooves extending into the interior space of the sphere is determined by the surface of the sphere, as is found to result from the base radius (4πr²).

The invention has realized that the amount of antibacterial active ingredient material dispensed via the surface of the material depot depends on the content of the active ingredient material in the carrier or depends on the loading of the carrier material with the active ingredient material, and that this content or load by the carrier material is subject to predefined upper limits. The invention has likewise realized that the antibiotic from the carrier material is only released from the uppermost layer of the carrier material. During examinations of explanted Septopal® chains, it could be verified that the antibiotic material gentamycin is only released from the outer region of the PMMA spheres, which is a few millimeters thick, while the deeper layers have unchanging proportions of antibiotics. Moreover, the invention has realized that increasing the surface of the material depot makes it possible to release more antibiotic per unit time, that is to say a higher level of antibiotics can be obtained. This makes it possible to counteract effectively under-dosage of the antibiotics. The high local concentration of antibiotics leads to an effective destruction of not only sensitive, but also of partially resistant germs.

The invention has likewise realized that the material depot according to the invention does not lead to a possible local overdose of the antibiotics in the region where it is implanted.

It was found that the antibiotic dispensed from the material depot to the wound fluid is distributed in the wound fluid by the dynamics of the wound fluid, for example by a change in the position of the patient or by muscular activity, and so an antibiotics level, which is reduced for the patient, is set. To the extent that an overdose cannot be excluded in an individual case, or if a delayed release of the antibiotic is desired, it is expedient to accordingly set the speed of the quantity of antibiotics dispensed. This can be effected by suitable proportions of the antibiotic in the material depot and/or of additives contained in the material depot which delay the release of the antibiotic. The speed of release can also be controlled by the material depot being compressed by different strengths or, for example, having a foam-like design.

The surface of the material depot can be increased by means of the most diverse surface structures, for example by depressions, hollow recesses, indentations and protuberances such as channels, folds, slits, lamellae, combs and bores. The channels, folds and slits run in the material depot, or on its surface, preferably in the longitudinal direction.

Advantageously, the surface structures do not damage, irritate or negatively affect the tissue in situ. Thus, the material depot advantageously does not have sharp edges. This is also advantageous when removing the material depot chain, described in more detail below, because damage to the tissue, the nerves and the vessels, and secondary hemorrhage can thus be avoided to the greatest possible extent when the chain is pulled out. Expediently, the surface structures, in particular the channels, folds and slits, run, at least in part, along the intended removal direction. In the case of Septopal® chains, which can be used in a technically developed form in accordance with claims 9 to 11, the removal direction, for example, is prescribed by the direction of the tensile force on the chain. The parallel arrangement of the channels, folds and slits according to the invention with respect to the intended direction of the tensile force supports the unimpeded slipping of the chain when being pulled out of the wound.

The depths or heights of the surface structures are expediently selected such that, on the one hand, the stiffness of the carrier material is not negatively affected and, for example, an undesired breaking-off of carrier material is avoided whilst implanting or explanting the material depot, or whilst repositioning the latter; on the other hand, the material thickness between the adjacent structures of the material depot is so thick that an active ingredient, which is as effective as possible, is dispensed from the carrier material in the wound. Thus, by way of example, lamellae, that is to say long sheets or blades, have a very large surface and stability measured in terms of the material expenditure, in particular when they are arranged in groups with their surfaces parallel or approximately parallel to each other and are connected to one another. If it is taken into account that the antibiotic material gentamycin is only released from the carrier material PMMA after the latter is implanted in the wound from an enveloping region which is a few millimeters thick, the material thickness between the adjacent structures of the material depot should preferably only be a few millimeters, preferably at most 5 mm, more preferably 3 mm, particularly preferably 1 to 3 mm.

The structuring of the material depot according to the invention permits the increase of the surface of the material depot compared to the envelope by at least a factor of 1.1, 1.2, 1.3, 1.4, 1.5, 1.6, 1.7, 1.8, 1.9 or by a multiplicity of times (by at least a factor of 2, 3, 4, 5, 6, 7, 8, 9, 10, 11-100, 100-200, 200-300, 300-400, 400-500, 500-600, 600-700, 700-800, 800-900 or 900-1000).

Expediently, the material with an antibacterial action is an antibiotic, gentamycin or a disinfecting substance being particularly expedient. As an alternative to, or in combination with, gentamycin, it is also possible to use further antibiotics, such as vanvomycin and/or clindamycin.

The material with an antibacterial action can be enclosed in a reabsorbable carrier material (e.g. polyglycolide-lactide or collagen) or a non-reabsorbable material (e.g. PMMA). Preferably, the material depot has, at least in parts, open pores on its surface. The open pores permit simplified dispensing of the antibacterial active ingredient material in the wound.

The basic geometric shape of the material depot, formed by the envelope, is preferably a shape selected from the group composed of cylinder, sphere, cone and the combination of these basic shapes such as the teardrop shape. In principle, every basic shape is preferred which permits, gently for the patient, implanting the material depot into the body and/or explanting it. “Gently” means in particular that the wound opening for implanting and/or explanting the material depot can be kept as small as possible, but at the same time nevertheless ensures simple removal.

Moreover, the subject matter of the present invention includes a material depot chain which has at least two material depots according to the invention as chain elements. The chain elements can be arranged on a central wire. The chain elements can, for example, have a spherical basic shape, analogous to the Septopal® chains known from the prior art, or the shape of a roller, for example in the shape of a solid cylinder. The adjacent chain elements can also have surfaces which correspond to each other. The surfaces can, for example, be of convex-concave design. This affords the possibility of an articulated movement of the chain elements which makes it easier to implant the chain, (re)position it if required or remove it, even in the case of a small distance between the adjacent chain elements. Such forms of chains have the advantage that they are flexible and can be matched to the local conditions of the wound.

### BRIEF DESCRIPTION OF THE DRAWINGS

In the following text, the invention will be described on the basis of advantageous embodiments with reference to the attached drawings, in which:

FIG. 1 shows a spherical material depot according to the invention comprising grooves;

FIG. 2 shows a material depot chain with spheres; and

FIG. 3 shows a material depot chain of a number of material depots in the shape of a roller with a convex/concave contact face design.

### DESCRIPTION OF THE INVENTION

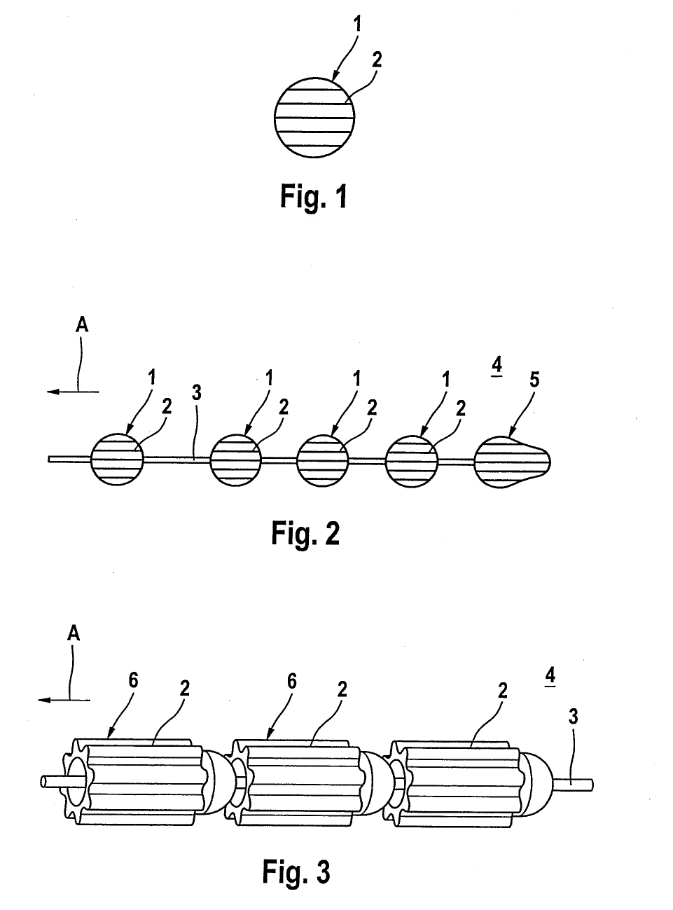

FIG. 1 shows a spherical material depot 1 with parallel grooves 2. The material depot 1 contains an antibacterial active ingredient material and dispenses the latter in a human or animal body (not illustrated) via its surface with open pores.

FIG. 2 shows a material depot chain 4 with a number of material depot spheres 1 in accordance with FIG. 1, which are fixed on a central wire 3. The material depots 1, 5 have grooves 2 which are arranged in parallel with respect to one another and in the direction of the tensile force in accordance with arrow A.

FIG. 3 illustrates a further material depot chain 4 with a number of roller-shaped material depots 6 which are fixed on a central wire 3. The roller-shaped material depots 6 have concave-convex faces which correspond to each other in place of the opposing planar surfaces which are customary in the case of a cylinder. This makes an articulated movement of the chain elements possible, even in the case of a small distance between the adjacent chain elements 6.

1. A material depot for dispensing an antibacterial active ingredient material in a human or animal body via a surface of the depot, wherein the surface of the material depot is larger than an envelope of the material depot.

2. The material depot of claim 1, wherein the surface of the material depot is larger than the envelope of the material depot by at least a factor of 1.1.

3. The material depot of claim 1 or 2, wherein the material depot has at least one surface structure selected from the group consisting of: depressions, hollow recesses, indentations and protuberances such as channels, folds, slits, lamellae, combs and bores.

4. The material depot of claim 3, wherein channels, folds and slits in the material depot run longitudinally, parallel or in a thread-like fashion.

5. The material depot of claim 1, wherein the antibacterial active ingredient material is enclosed in at least one of a carrier material intended to be reabsorbed by the body and a carrier material is not intended to be reabsorbed by the body.

6. The material depot of claim 1, wherein the material thickness between adjacent structures of the material depot does not exceed 5 mm.

7. The material depot of claim 1, wherein the material depot has, at least in parts, open pores on its surface.

8. The material depot of claim 1, wherein the material depot is shaped as a cylinder, sphere, cone, or combination of these basic shapes such as a teardrop shape.

9. A material depot chain comprising, as chain elements, at least two material depots according to claim 1.

10. The material depot chain of claim 9, wherein opposing faces of adjacent material depot chain elements have a corresponding shape, at least in part.

11. The material depot chain of claim 10, wherein the opposing faces of the adjacent material depot chain elements have a concave-convex design.

12. The material depot of claim 1, wherein the surface of the material depot is larger than the envelope of the material depot by at least a factor of 2.

13. The material depot of claim 1, wherein the surface of the material depot is larger than the envelope of the material depot by at least a factor of 3.

14. The material depot of claim 6, wherein the material thickness between the adjacent structures of the material depot does not exceed 3 mm.

15. The material depot of claim 14, wherein the material thickness between the adjacent structures of the material depot is between 1 and 3 mm.
